(19) United States
(12) Patent Application Publication
(10) Pub. No.: US 2019/0120819 A1
(43) Pub. Date: Apr. 25, 2019

(54) BLOOD ANALYSIS DEVICE

(71) Applicants: ELECTRONICS AND TELECOMMUNICATIONS RESEARCH INSTITUTE, Daejeon (KR); KOREA INSTITUTE OF ORIENTAL MEDICINE, Daejeon (KR)

(72) Inventors: Hyung Ju PARK, Suwon (KR); Moon Youn JUNG, Daejeon (KR); Dae-Sik LEE, Daejeon (KR); Ju Ah LEE, Daejeon (KR); Jeeyoun JUNG, Iksan (KR)

(21) Appl. No.: 16/165,905

(22) Filed: Oct. 19, 2018

(30) Foreign Application Priority Data
Oct. 19, 2017 (KR) 10-2017-0136103
Aug. 7, 2018 (KR) 10-2018-0091957

(51) Int. Cl. G01N 33/49; G01N 21/31; G01N 11/00

(52) U.S. Cl. CPC G01N 33/49; G01N 2011/008; G01N 11/00; G01N 21/3151

(57) ABSTRACT

Provided is a blood analysis device including a capillary tube, a photo sensor disposed on a sidewall of the capillary tube to detect blood flowing in the capillary tube, and an absorption sensor coupled to one end of the capillary tube to absorb the blood in the capillary tube.

### CROSS-REFERENCE TO RELATED APPLICATIONS

This U.S. non-provisional patent application claims priority under 35 U.S.C. § 119 of Korean Patent Application No. 10-2017-0136103, filed on Oct. 19, 2017, and 10-2018-0091957, filed on Aug. 07, 2018, the entire contents of which are hereby incorporated by reference.

### BACKGROUND

The present disclosure herein relates to a blood analysis device, and more particularly, to a blood analysis device capable of detecting various bio-markers.

When blood is analyzed, a health condition, presence or absence of a disease, or the like may be diagnosed. Bio-markers of blood, which is an object to be analyzed, include physical characteristics such as viscosity and hematocrit, chemical characteristics such as blood glucose, and biological characteristics such as an immune substance. The viscosity indicates a degree of resistance to flow of fluid. In case of blood, when the viscosity is deviated from a predetermined range, the blood is considered to be abnormal. The hematocrit represents a volume ratio of red blood cells to entire blood. The hematocrit may be used for diagnosis of anemia or the like. The blood glucose indicates glucose contained in blood. When the concentration of blood glucose is measured, the presence or absence of a disease such as diabetes may be diagnosed. The immune substance is a material for protecting internal environment of a human body from antigen that is an external factor. The immune substance may be measured through various immune reactions. Besides, various physical/chemical/biological characteristics are necessary to be measured for recognizing a health condition of the human body.

### SUMMARY

The present disclosure provides a blood analysis device capable of measuring physical/chemical/biological characteristics at once.

The present disclosure also provides a blood analysis device capable of exactly and rapidly measuring various bio-markers while reducing a volume thereof.

The object of the present disclosure is not limited to the aforesaid, but other objects not described herein will be clearly understood by those skilled in the art from descriptions below.

An embodiment of the inventive concept provides a blood analysis device including: a capillary tube; a photo sensor disposed on a sidewall of the capillary tube to detect blood flowing in the capillary tube; and an absorption sensor coupled to one end of the capillary tube to absorb the blood in the capillary tube.

In an embodiment, the absorption sensor may contain a sensing material configured to measure chemical or biological characteristics.

In an embodiment, the blood analysis device may further include: a control unit; and a reader configured to detect variation of the absorption sensor, and the reader may transmit information on the variation of the absorption sensor to the control unit.

In an embodiment, the photo sensor may measure a transmittance of the blood flowing in the capillary tube.

In an embodiment, the blood analysis device may further include a control unit, and the control unit may receive information on a transmittance of the blood from the photo sensor to calculate a viscosity of the blood flowing in the capillary tube.

In an embodiment, the blood analysis device may further include a temperature sensor, and the control unit may receive information on a temperature of the blood from the temperature sensor to correct the viscosity of the blood.

In an embodiment, the blood analysis device may further include a rotation guide; and a rotation support, and the capillary tube may be coupled to the rotation support so as to rotate along the rotation guide.

In an embodiment, the blood analysis device may further include a display unit, and the control unit may display the viscosity of the blood on the display unit.

In an embodiment, the capillary tube may contain a transparent material.

In an embodiment, the capillary tube may have an inner surface that is surface-treated by EDTA or heparin.

In an embodiment, the capillary tube may have an inside diameter of about 1 mm or less.

In an embodiment of the inventive concept, a blood analysis device includes: a capillary tube; a first photo sensor; a second photo sensor; and a control unit. Each of the first photo sensor and the second photo sensor is disposed on a sidewall of the capillary tube to measure a transmittance of blood flowing in the capillary tube, and the control unit receives information on the transmittance of the blood from the first and second photo sensors to calculate hematocrit (HCT) of the blood flowing in the capillary tube.

In an embodiment, the blood analysis device may further include a0 control unit, and the control unit may receive the information on the transmittance of the blood from the first and second photo sensors to calculate a viscosity of the blood flowing in the capillary tube.

In an embodiment, the first photo sensor may include a first light emitting part and a first light receiving part, the second photo sensor may include a second light emitting part and a second light receiving part, and the first light emitting part and the second light emitting part may emit light having wavelengths different from each other.

In an embodiment, the first light emitting part may emit first light, the second light emitting part may emit second light, the first light may have a wavelength of about 800 to about 1000 nm, and the second light may have a wavelength of about 500 to about 600 nm.

In an embodiment, the blood analysis device may further include an absorbent agent, and the absorbent agent may be coupled to one end of the capillary tube to absorb the blood in the capillary tube.

The objects of the present disclosure are not limited to the aforementioned object, but other objects not described herein will be clearly understood by those skilled in the art from descriptions below.

### BRIEF DESCRIPTION OF THE FIGURES

The accompanying drawings are included to provide a further understanding of the inventive concept, and are incorporated in and constitute a part of this specification. The drawings illustrate exemplary embodiments of the inventive concept and, together with the description, serve to explain principles of the inventive concept. In the drawings:

FIG. 1 is a front view illustrating a blood analysis device according to an exemplary embodiment of the inventive concept;

FIG. 2 is a front view illustrating an operation principle of the blood analysis device according to an exemplary embodiment of the inventive concept;

FIG. 3 is a conceptual view illustrating a control flow of the blood analysis device according to an exemplary embodiment of the inventive concept;

FIG. 4 is a flowchart illustrating an operation sequence of the blood analysis device according to an exemplary embodiment of the inventive concept;

FIG. 5 is a front view illustrating an initial state of the blood analysis device in which blood is filled according to an exemplary embodiment of the inventive concept;

FIG. 6 is a front view illustrating a state in which blood in the blood analysis device is dropped to pass a first photo sensor according to an exemplary embodiment of the inventive concept;

FIG. 7 is a front view illustrating a state in which blood in the blood analysis device is dropped to pass a second photo sensor according to an exemplary embodiment of the inventive concept;

FIG. 8 is a front view illustrating a state in which blood in the blood analysis device is dropped to pass a third photo sensor according to an exemplary embodiment of the inventive concept; and

FIG. 9 is a front view illustrating a blood analysis device according to another embodiment of the inventive concept.

### DETAILED DESCRIPTION

Exemplary embodiments of technical ideas of the inventive concept will be described with reference to the accompanying drawings so as to sufficiently understand constitutions and effects of the inventive concept. The present disclosure may, however, be embodied in different forms and should not be construed as limited to the embodiments set forth herein. Rather, these embodiments are provided so that this disclosure will be thorough and complete, and will fully convey the scope of the present disclosure to those skilled in the art. Further, the present disclosure is only defined by scopes of claims.

Like reference numerals refer to like elements throughout. The embodiment in the detailed description will be described with cross-sectional views and/or plan views as ideal exemplary views of the inventive concept. In the figures, the dimensions of regions are exaggerated for effective description of the technical contents. Areas exemplified in the drawings have general properties, and are used to illustrate a specific shape of a semiconductor package region. Thus, this should not be construed as limited to the scope of the present disclosure. It will be understood that although the terms first and second are used herein to describe various elements, these elements should not be limited by these terms. These terms are only used to distinguish one component from another component. Embodiments described and exemplified herein include complementary embodiments thereof.

In the following description, the technical terms are used only for explaining a specific exemplary embodiment while not limiting the present disclosure. In this specification, the terms of a singular form may include plural forms unless specifically mentioned. The meaning of “include,” “comprise,” “including,” or “comprising,” specifies a property, a region, a fixed number, a step, a process, an element and/or a component but does not exclude other properties, regions, fixed numbers, steps, processes, elements and/or components.

Hereinafter, the present disclosure will be described in detail by explaining preferred embodiments of the disclosure with reference to the attached drawings.

Large-sized equipment may be necessary to measure various bio-markers. Such equipment may be inconvenient because the equipment has a large volume and great amount of samples (blood) is required. For example, a centrifugal separator is necessary to measure hematocrit. Since the centrifugal separator is heavy and expensive, the centrifugal separator may be difficult to be used at a site. Accordingly, when samples are transferred to analysis equipment after sampling, much time may be required. Furthermore, when the number of samples to be analyzed is great, more time may be required. Also, since one equipment may detect only one bio-marker, a plurality of equipment are necessary to detect a plurality of bio-markers. Thus, analysis equipment capable of instantly analyzing various bio-markers at a site after blood sampling is required to resolve the above-described limitations.

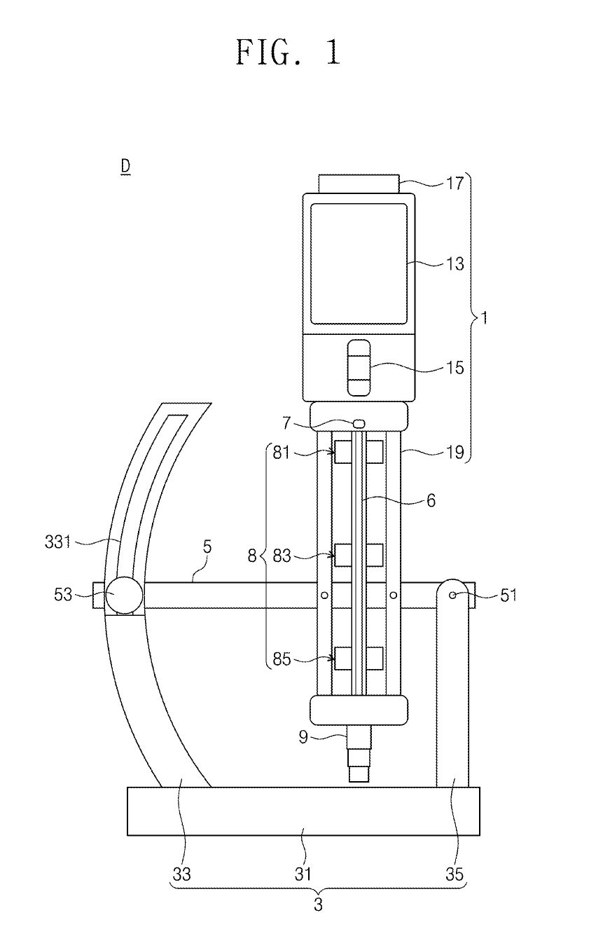

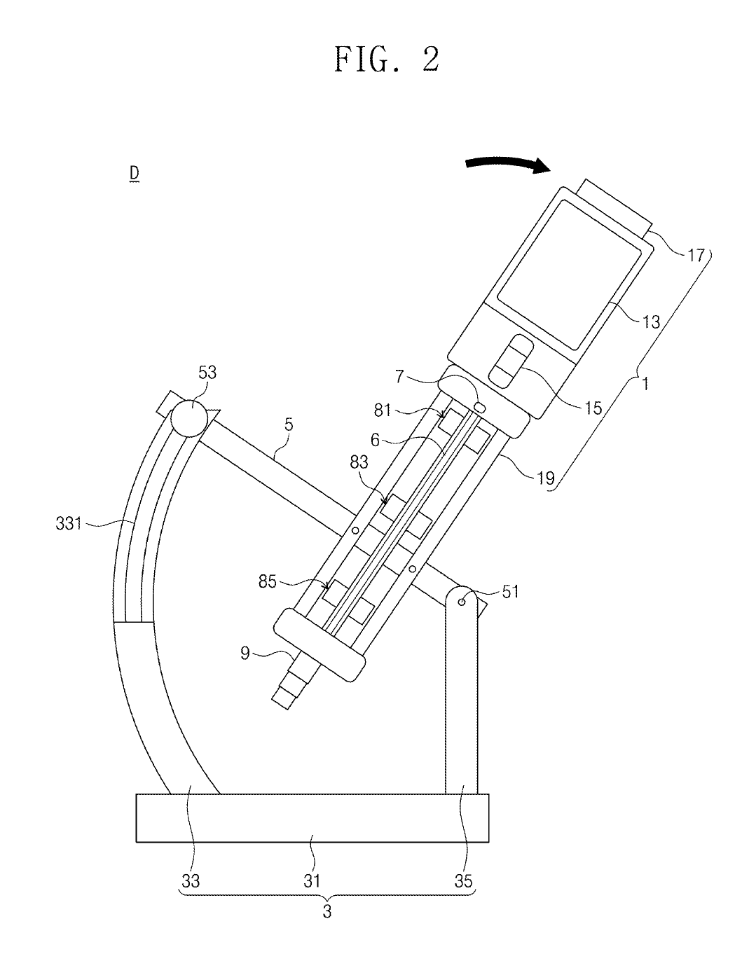

FIG. 1 is a front view illustrating a blood analysis device according to an exemplary embodiment of the inventive concept, and FIG. 2 is a front view illustrating a mechanical operation process of the blood analysis device according to an exemplary embodiment of the inventive concept.

Referring to FIG. 1, a blood analysis device D may include a capillary tube 6, a photo sensor 8, an absorption sensor 9, a body 1, a cradle 3, a connecting part 5, and a temperature sensor 7.

The capillary tube 6 may have a stick shape. The capillary tube 6 may be vertically spread. The capillary tube 6 may include an inner passage through which blood may flow. Although the inner passage of the capillary tube 6 has a circular cross-section, the embodiment of the inventive concept is not limited thereto. The capillary tube 6 may contain a transparent material. The blood flowing in the capillary tube 6 may be observed from the outside of the capillary tube 6. Although disposed at the outside of the capillary tube 6, the photo sensor 8 may observe the inside of the capillary tube 6. In an exemplary embodiment, the inner passage of the capillary tube 6 may have an inside diameter equal to or less than about 1 mm, and the capillary tube 6 may have a length equal to or less than about 10 cm. However, the embodiment of the inventive concept is not limited thereto. For example, the inner passage of the capillary tube 6 may have a different inside diameter as long as the blood flows by a capillary phenomenon, and the capillary tube 6 may have a length greater than about 10 cm. The capillary tube 6 may have an inner surface that is surface-treated by using a material preventing the blood from being coagulated. In an exemplary embodiment, the inner surface of the capillary tube 6 may be surface-treated by using ethylenediaminetetraacetic acid (EDTA) or heparin. Accordingly, the blood flowing in the capillary tube 6 may be prevented from being coagulated.

The photo sensor 8 may be disposed adjacent to a sidewall of the capillary tube 6. The photo sensor 8 may detect the blood flowing in the capillary tube 6. The photo sensor 8 may emit an electromagnetic wave toward the blood and/or the capillary tube 6 and detect the electromagnetic wave, which has passed through the blood and/or the capillary tube 6. The photo sensor 8 may include a first photo sensor 81, a second photo sensor 83, and a third photo sensor 85.

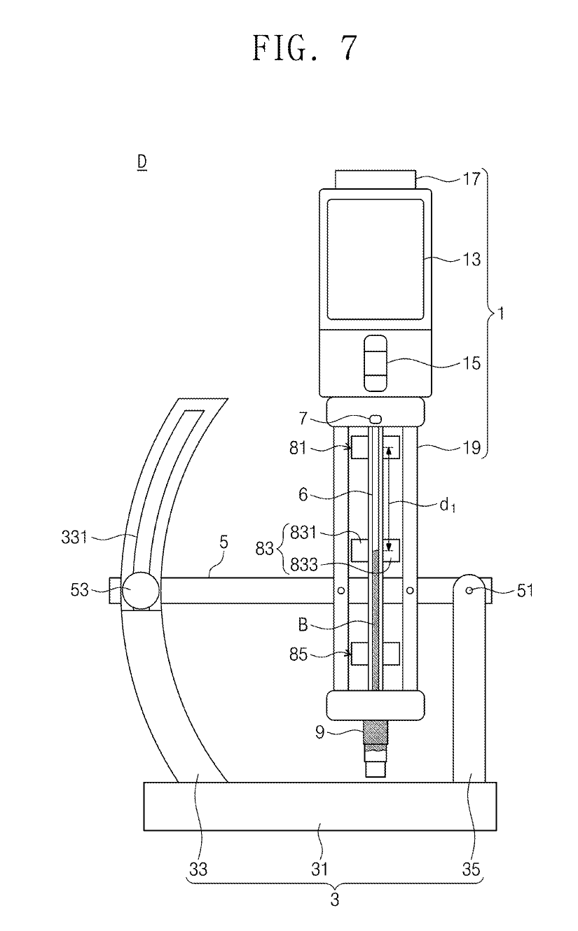

The first photo sensor 81 may include a first light emitting part 811 and a first light receiving part 813. The first light emitting part 811 and the first light receiving part 813 may face each other with the capillary tube 6 therebetween. The first light emitting part 811 may emit first light toward the first light receiving part 813. The first light receiving part 813 may detect the first light emitted from the first light emitting part 811. The first photo sensor 81 may be spaced by dl from the second photo sensor 83. A detailed configuration regarding this will be described later with reference to FIG. 7.

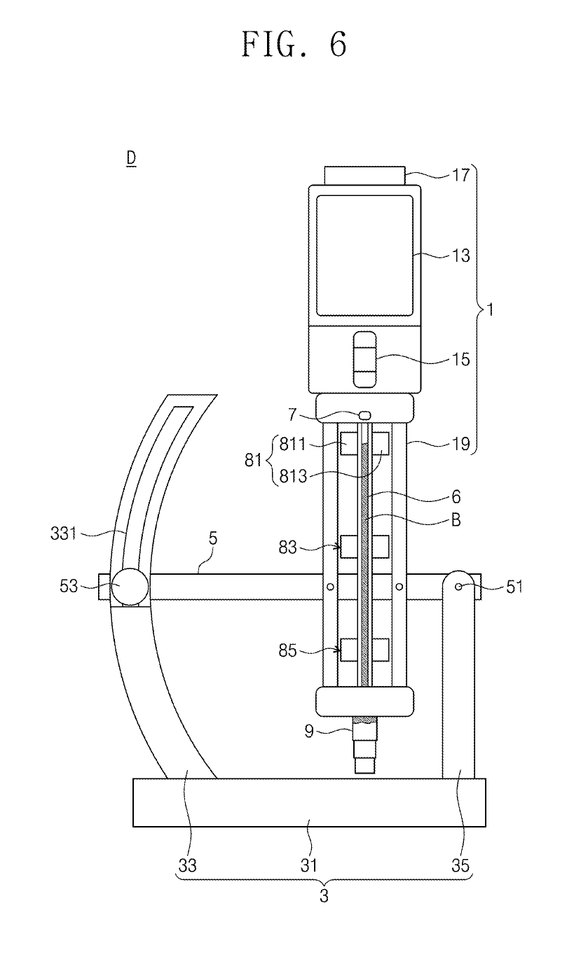

The second photo sensor 83 may include a second light emitting part 831 and a second light receiving part 833. The second light emitting part 831 and the second light receiving part 833 may face each other with the capillary tube 6 therebetween. The second light emitting part 831 may emit second light toward the second light receiving part 833. The second light receiving part 833 may detect the second light emitted from the second light emitting part 831. The second photo sensor 83 may be spaced by d2 from the third photo sensor 85. A detailed configuration regarding this will be described later with reference to FIG. 6.

The third photo sensor 85 may include a third light emitting part 851 and a third light receiving part 853. The third light emitting part 851 and the third light receiving part 853 may face each other with the capillary tube 6 therebetween. The third light emitting part 851 may emit third light toward the third light receiving part 853. The third light receiving part 853 may detect the third light emitted from the third light emitting part 851.

The first light may have a wavelength different from that of the second light. In an exemplary embodiment, the first light may have a wavelength of about 800 nm to about 1000 nm. The second light may have a wavelength of about 500 nm to about 600 nm. More preferably, the wavelength of the first light may be about 880 nm, and the wavelength of the second light may be about 532 nm. However, the embodiment of the inventive concept is not limited thereto. For example, each of the first and second light may have a different wavelength, which satisfies the purpose of an embodiment of the inventive concept.

The absorption sensor 9 may detect various bio-markers of the blood. The absorption sensor 9 may contain a material such as paper. In an exemplary embodiment, the absorption sensor 9 may include a strip sensor. The absorption sensor 9 may be coupled to one end of the capillary tube 6. The absorption sensor 9 may absorb blood. The absorption sensor 9 may be coated with a sensing material that generates a chemical/biological reaction with a specific material in blood. The sensing material may include various materials generating a reaction with a specific material in blood. In an exemplary embodiment, the absorption sensor 9 may be coated with a blood glucose sensing material generating a chemical reaction with the blood glucose in blood. The absorption sensor 9 may be coated with an immune sensing substance generating a biological reaction with an immune substance in blood. The immune substance and the immune sensing substance may generate an immune reaction. The immune reaction may represent an antigen-antibody immune reaction including a C-reactive protein (CRP) test. However, the embodiment of the inventive concept is not limited thereto. For example, various sensing materials capable of detecting various chemical/biological characteristics in blood may be applied. In an exemplary embodiment, the absorption sensor 9 may be coated with only one of the various sensing materials. The absorption sensor 9 may detect only one of specific materials in blood. In another exemplary embodiment, the absorption sensor 9 may be coated with two or more of the various sensing materials. The absorption sensor 9 may be divided into several sections to contain various sensing materials. Alternatively, the absorption sensor 9 may be coated with various sensing materials sequentially along a longitudinal direction. Various chemical/biological materials in blood may be detected at once by one absorption sensor 9.

When blood, which is absorbed by absorbing force of the absorption sensor 9, contacts the sensing material applied on the absorption sensor 9, the chemical or biological reaction may be generated. A portion of the absorption sensor 9, on which a material is applied, may be changed in color. In an exemplary embodiment, the absorption sensor 9 may be changed in color by generating a chemical reaction with the blood glucose in blood. The absorption sensor 9 may be changed in color by generating a biological reaction with the immune substance in blood. Besides, absorption sensor 9 may be changed in color by reacting with various materials in blood.

The absorption sensor 9 may detect various bio-markers in blood. The absorption sensor 9 may extend in a longitudinal direction of the capillary tube 6. The absorbing force of the absorption sensor 9 may move the blood in the capillary tube 6.

In an exemplary embodiment, the blood analysis device D may further include a reader for detecting the absorption sensor 9. The reader may detect whether the sensing material applied on the absorption sensor 9 reacts with the specific material in the blood. In an exemplary embodiment, the reader may detect color change of the absorption sensor 9. However, the embodiment of the inventive concept is not limited thereto. For example, a user may notice a state of the specific material in the blood by checking the color or the like of the absorption sensor 9 through naked eyes.

The body 1 may support the capillary tube 6 or the like. The body 1 may include a support bar 19, a display unit 13, a manipulation unit 15, and a charging terminal 17.

The support bar 19 may support the capillary tube 6 and the photo sensor 8. In an exemplary embodiment, the support bar 19 may include two bars each extending in the longitudinal direction of the capillary tube 6. However, the embodiment of the inventive concept is not limited thereto. For example, the support bar 19 has a shape supporting the capillary tube 6 and the photo sensor 8.

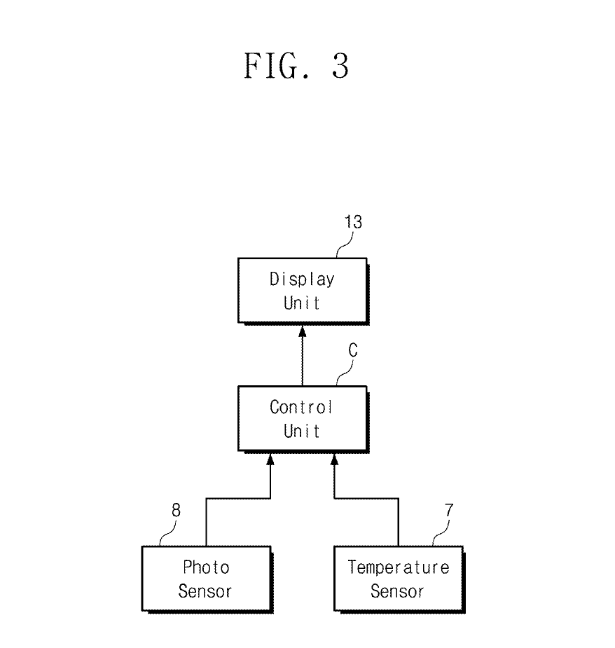

The display unit 13 may display a bio-marker of the blood, which is measured by the photo sensor 8. The display unit 13 may receive information on the blood from the control unit C (refer to FIG. 3). Detailed description regarding this will be described later.

The manipulation unit 15 may supply blood to the capillary tube 6 or discharge the blood filled in the capillary tube 6. The manipulation unit 15 may control the control unit C.

The charging terminal 17 may be connected to an external power. The external power may supply power to the display unit 13, the control unit C, or the like through the charging terminal 17. Although the charging terminal 17 may be disposed on an upper end of the body 1, the embodiment of the inventive concept is not limited thereto.

The cradle 3 may support the entire blood analysis device D including the body 1. The cradle 3 may include a stand 31, a rotation guide 33, and a rotation support 35.

The stand 31 may have a plate shape. The stand 31 may support the blood analysis device D. The rotation guide 33 and the rotation support 35 may be coupled to a top surface of the stand 31.

The rotation guide 33 may have an arc shape extending upward from the top surface of the stand 31. The rotation guide 33 may include a slide hole 331. The slide hole 331 may be a hole extending in an arc shape centered about one end of the rotation support 35. More specifically, the slide hole 331 may have an arc shape centered about a pivot 51. A slider 53 may be inserted into the slide hole 331. The slide hole 331 and the slider 53 may be coupled to each other in a slidable manner.

The rotation support 35 may extend upward from a top surface of the stand 31. The rotation support 35 may support rotation of the connecting part 5. The rotation support 35 may have one end, about which the connecting part 5 rotates.

The connecting part 5 may connect the body 1 to the cradle 3. The connecting part 5 may be fixed to the body 1. More specifically, the connecting part 5 may be coupled to the support bar 19. The connecting part 5 may include the pivot 51 and the slider 53. The connecting part 5 may be coupled to the rotation support 35 in a rotatable manner by the pivot 51. The connecting part 5 may be coupled to the slide hole 331 in a slidable manner by the slider 53. Referring to FIG. 2, the connecting part 5 rotates about the pivot 51 in a clockwise direction, so that the capillary tube 6 is inclined to the right side. The connecting part 5 may continue to rotate until the slider 53 contacts one end of the slide hole 331. An angle formed between the capillary tube 6 and the stand 31 may be variously changed. Also, a speed of the blood flowing in the capillary tube 6 may be variously changed.

The temperature sensor 7 may measure a temperature of the blood flowing in the capillary tube 6. The temperature sensor 7 may transmit information on the measured temperature to the control unit C.

FIG. 3 is a schematic view illustrating a control flow of the blood analysis device according to an exemplary embodiment of the inventive concept.

Referring to FIG. 3, the blood analysis device D may further include a control unit C.

The control unit C may receive information on a transmittance of blood and information on a time at which the transmittance is varied from the photo sensor 8. The control unit C may calculate the speed of the blood passing through the capillary tube 6 on the basis of the information on the transmittance of the blood and the variation time of the transmittance.

The control unit C may measure hamatocrit (CT) of the blood on the basis of the information on the transmittance of the blood, which is measured by the first photo sensor 81 and the second photo sensor 83.

The control unit C may receive information on a temperature of the blood in the capillary tube 6 from the temperature sensor 7. The control unit C may correct a viscosity of the blood on the basis of the information on the temperature of the blood, which is received from the temperature sensor 7.

The control unit C may be electrically connected to the reader. The control unit C may receive information on variation of the absorption sensor 9 from the reader. The control unit C may recognize whether a specific material exists in the blood absorbed by the absorption sensor 9 or an amount of a specific material.

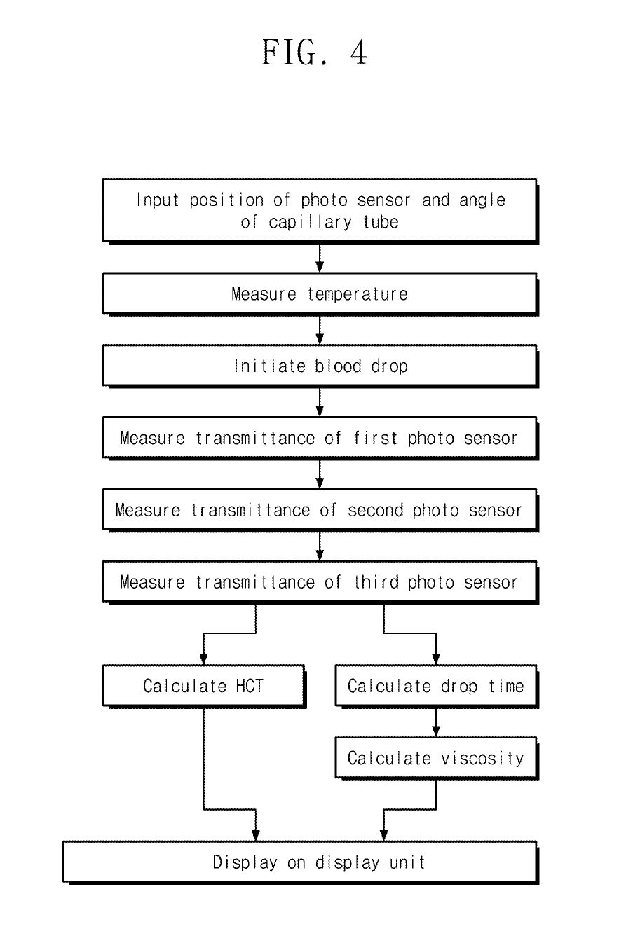

FIG. 4 is a flowchart illustrating an operation sequence of the blood analysis device in detail according to an exemplary embodiment of the inventive concept, and FIGS. 4 to 8 are front views illustrating an operation principle of the blood analysis device in detail according to an exemplary embodiment of the inventive concept.

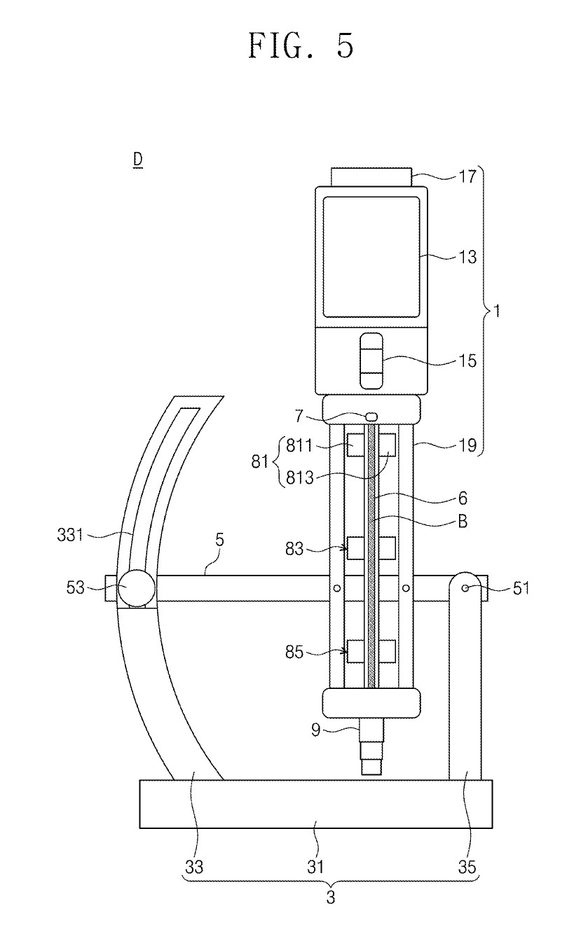

Referring to FIGS. 4 and 5, the control unit C may receive information including a position of the photo sensor 8 and an angel formed between the capillary tube 6 and the ground or the stand 31. The capillary tube 6 may be filled with blood B. The temperature sensor 7 may measure a temperature of the blood B in the capillary tube 6. The control unit C may receive information on the temperature of the blood B from the temperature sensor 7. The viscosity of fluid may be a function of temperature. When the control unit C calculate the viscosity of the blood B, the temperature of the blood B may be used to obtain more exact value. When the absorption sensor 9 is coupled to one end of the capillary tube 6, the absorption sensor 9 may absorb the blood B. The blood B may start to be absorbed to the absorption sensor 9. The blood B may be dropped toward the absorption sensor 9 by the absorbing force of the absorption sensor 9, the gravity acting on the blood B, and the capillary phenomenon of the capillary tube 6.

The first light emitting part 811 may emit first light toward the blood B in the capillary tube 6. The first light may pass through the blood B and be detected by the first light receiving part 813. The first light receiving part 813 may detect a light absorption rate of the blood B with respect to the first light. The first light receiving part 813 may transmit the light absorption rate of the blood B with respect to the first light to the control unit C.

The second light emitting part 831 may emit second light toward the blood B in the capillary tube 6. The second light may pass through the blood B and be detected by the second light receiving part 833. The first light receiving part 813 may detect a light absorption rate of the blood B with respect to the second light. The second light receiving part 833 may transmit the light absorption rate of the blood B with respect to the second light to the control unit C.

The first light and the second light may have wavelengths different from each other. In an exemplary embodiment, the wavelength of the first light may be about 800 nm to about 1000 nm. In an exemplary embodiment, the wavelength of the first light may be about 500 nm to about 600 nm. The control unit C may calculate hematocrit of the blood B on the basis of the light absorption rate of the blood B with respect to the first light and the light absorption rate of the blood B with respect to the second light. The control unit C may transmit information on the calculated hematocrit to the display unit 13. The display unit 13 may display the information on the hematocrit of the blood B. According to an exemplary embodiment of the inventive concept, the display unit 13 may separately display a case of a normal range and a case of an abnormal range of the hematocrit of the blood. That is, the control unit C may transmit a signal representing a normal state to the display unit 13 when the measured hematocrit of the blood is within the normal range and a signal representing an abnormal state to the display unit 13 when the measured hematocrit of the blood is within the abnormal range. In an exemplary embodiment, the display unit 13 may display whether the hematocrit of the blood is within the normal range or the abnormal range by using an icon such as a drawing. The user may rapidly recognize a blood health condition by seeing the normal/abnormal signal, which is displayed on the display unit 13. However, the embodiment of the inventive concept is not limited thereto. For example, the hematocrit may be expressed in specific numbers.

Referring to FIGS. 4 and 6, as the blood B is dropped, an upper boundary surface of the blood B may pass through the first photo sensor 81. The first light emitted from the first light emitting part 811 may be arrived to the first light receiving part 813 without passing through the blood B. The first light detected by the first light receiving part 813 may be varied in state. A time (t1) at which the state of the first light detected by the first light receiving part 813 is varied may be a time at which the upper boundary surface of the blood B passes the first photo sensor 81. Information on variation of the first light detected by the first light receiving part 813 may be transmitted to the control unit C. The control unit C may store the time (t1) at which the upper boundary surface of the blood B passes the first photo sensor 81.

Referring to FIGS. 4 and 7, as the blood B is dropped, the upper boundary surface of the blood B may pass through the second photo sensor 83. The second light emitted from the second light emitting part 831 may be arrived to the second light receiving part 833 without passing through the blood B. The second light detected by the second light receiving part 833 may be varied in state. A time (t2) at which the state of the second light detected by the second light receiving part 833 is varied may be a time at which the upper boundary surface of the blood B passes the second photo sensor 83. Information on variation of the second light detected by the second light receiving part 833 may be transmitted to the control unit C. The control unit C may store the time (t2) at which the upper boundary surface of the blood B passes the second photo sensor 83.

The control unit C may be inputted with a distance dl between the first photo sensor 81 and the second photo sensor 83. The control unit C may calculate a moving speed of the blood B by using the distance dl between the first photo sensor 81 and the second photo sensor 83 and a time (t2-t1) during which the upper boundary surface of the blood B moves from the first photo sensor 81 to the second photo sensor 83. More specifically, the moving speed of the blood B may be a value obtained by dividing dl by (t2-t1). The moving speed value of the blood B, which is obtained by the calculation, may be referred to as v1.

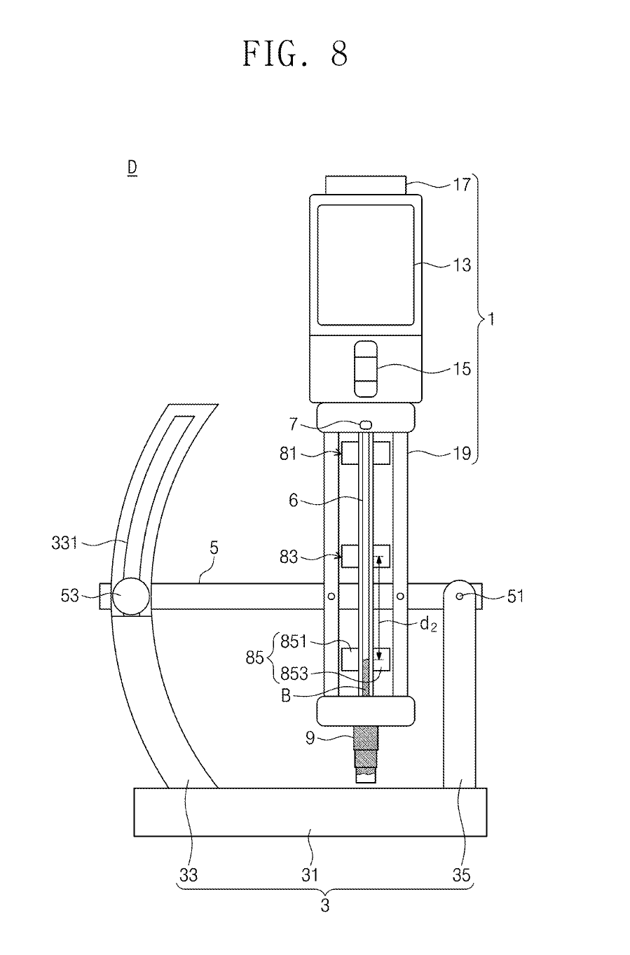

Referring to FIGS. 4 and 8, as the blood B is dropped, the upper boundary surface of the blood B may pass through the third photo sensor 85. The third light emitted from the third light emitting part 851 may be arrived to the third light receiving part 853 without passing through the blood B. The third light detected by the third light receiving part 853 may be varied in state. A time (t3) at which the state of the third light detected by the third light receiving part 853 is varied may be a time at which the upper boundary surface of the blood B passes the third photo sensor 85. Information on variation of the third light detected by the third light receiving part 853 may be transmitted to the control unit C. The control unit C may store the time (t3) at which the upper boundary surface of the blood B passes the third photo sensor 85.

The control unit C may be inputted with a distance d2 between the second photo sensor 83 and the third photo sensor 85. The control unit C may calculate a moving speed of the blood B by using the distance d2 between the second photo sensor 83 and the third photo sensor 85 and a time (t3-t2) during which the upper boundary surface of the blood B moves from the second photo sensor 83 to the third photo sensor 85. More specifically, the moving speed of the blood B may be obtained by dividing d2 by (t3-t2). The moving speed value of the blood B, which is obtained by the calculation, may be referred to as v2.

The control unit C may calculate the moving speed of the blood B in a more exact manner by using the three photo sensors 81, 83, and 85. In an exemplary embodiment, the moving speed of the blood B may be a mean value of v1 and v2.

The control unit C may calculate the viscosity of the blood B by using the moving speed (v1, v2, or the mean value of v1 and v2) of the blood.

In an exemplary embodiment, the control unit C may calculate a force applied to the blood from an inside diameter of the capillary tube 6, a length of the capillary tube 6, a force caused by a capillary phenomenon, an angle formed between the capillary tube 6 and the ground or the stand 31, gravity acting on the blood, and an absorption force of the absorption sensor 9, calculate a speed distribution of the blood from the inside diameter of the capillary tube 6 and the moving speed of the blood, and calculate the viscosity of the blood by using the force applied to the blood and the speed distribution of the blood. That is, since a flow rate of the blood flowing in the capillary tube 6 is proportional to a pressure difference between both ends of the capillary tube 6 and inversely proportional to the viscosity, the viscosity of the blood may be calculated by obtaining the flow rate of the blood from the inside diameter of the capillary tube 6 and the moving speed of the blood and the pressure difference between the both ends of the capillary tube 6 from the inside diameter of the capillary tube 6 and the force applied to the blood.

When calculates the viscosity of the blood, the control unit C may use the temperature of the blood for more accurate calculation. A viscosity of fluid may be affected by a temperature. When the temperature of the blood is changed, the viscosity of the blood may be changed. When the blood temperature when the viscosity is measured is different from the blood temperature in a normal body temperature, the health condition may be properly determined. The control unit C may receive information on the blood temperature from the temperature sensor 7. The control unit C may correct the viscosity of the blood calculated by using the temperature of the blood at the measurement time into the viscosity of the blood in the normal body temperature. Accordingly, the control unit C may exactly diagnose the blood health condition.

The control unit C may transmit information on the viscosity of the blood to the display unit 13. The display unit 13 may display the information on the viscosity of the blood. According to an exemplary embodiment of the inventive concept, the display unit 13 may separately display a case of a normal range and a case of an abnormal range of the viscosity of the blood. That is, the control unit C may transmit a signal representing a normal state to the display unit 13 when the measured viscosity of the blood is within the normal range and a signal representing an abnormal state to the display unit 13 when the measured viscosity of the blood is within the abnormal range. In an exemplary embodiment, the display unit 13 may display whether the viscosity of the blood is within the normal range or the abnormal range by using an icon such as a drawing. The user may rapidly recognize a blood health condition by seeing the normal/abnormal signal, which is displayed on the display unit 13. However, the embodiment of the inventive concept is not limited thereto. For example, the viscosity may be expressed in specific numbers.

In an exemplary embodiment, when the moving speed of the blood is excessively fast or excessively slow, an inclination of the body 1 may be varied to achieve a proper speed. That is, when a meaningful measurement value is hard to be obtained because of the excessively fast speed of the blood, the body 1 may be inclined as illustrated in FIG. 2. The capillary tube 6 also may be inclined. Accordingly, the speed of the blood may become slow. The control unit C may be inputted with a value regarding the inclination of the capillary tube 6. The control unit C may consider the inclination of the capillary tube 6 when the viscosity of the blood is measured. When fast measurement is difficult because of the excessively slow speed of the blood, the body may stand almost vertically. The capillary tube 6 also may stand. Accordingly, the speed of the blood may become fast. The control unit C may be inputted with a value regarding the inclination of the capillary tube 6. The control unit C may consider the inclination of the capillary tube 6 when the viscosity of the blood is measured. As the inclination of the capillary tube 6 is variously changed, proper values may be ensured when the viscosity of the blood is measured. As the inclination of the capillary tube 6 is variously changed, a fast operation may be performed when the viscosity of the blood is measured.

Referring to FIGS. 6 to 8, the blood B in the capillary tube 6 may be absorbed to the absorption sensor 9. The absorption sensor 9 may contain a sensing material for determining whether a specific material exists in the blood B or measuring an amount of the specific material. In an exemplary embodiment, the absorption sensor 9 may be coated with a blood glucose sensing material generating a chemical reaction with the blood glucose in the blood. The absorption sensor 9 may be coated with an immune sensing substance generating a biological reaction with an immune substance in the blood. The immune substance and the immune sensing substance may generate an immune reaction. The immune reaction may represent an antigen-antibody immune reaction including a C-reactive protein (CRP) test. However, the embodiment of the inventive concept is not limited thereto. For example, various sensing materials capable of detecting various chemical/biological characteristics in the blood may be applied.

When the blood B contacts the sensing material on the absorption sensor 9, the chemical reaction or the biological reaction may be generated. Due to the chemical reaction or the biological reaction, the characteristics of the absorption sensor 9 may be changed. In an exemplary embodiment, the absorption sensor 9 may be changed in color. Through the color change of the absorption sensor 9, the condition of the blood B may be diagnosed.

The control unit C may receive information on variation of the absorption sensor 9 from the reader. The control unit C may recognize whether a specific material exists in the blood or the amount of the specific material from the variation of the absorption sensor 9. The control unit C may transmit information on whether a specific material exists in the blood or the amount of the specific material to the display unit 13. The display unit 13 may display the information on whether a specific material exists in the blood or the amount of the specific material. The user may rapidly recognize the blood condition by seeing the display unit 13. However, the embodiment of the inventive concept is not limited thereto. For example, the user may directly detect the color change to recognize the blood condition.

The absorption sensor 9 may provide an absorption force for absorbing the blood B. When the absorption sensor 9 is used, an additional pump may be unnecessary. The absorption sensor 9 may be inexpensive. Accordingly, when the absorption sensor 9 is used, the blood analysis device D may become inexpensive. The absorption sensor 9 may have a small volume. Accordingly, when the absorption sensor 9 is used, the blood analysis device D may become smaller in volume. When the absorption sensor 9 is used, the measurement of the viscosity and/or hematocrit of the blood and the measurement of other chemical/biological characteristics may be performed together. The absorption sensor 9 may be disposable. The absorption sensor 9 may be discarded after the condition of the blood B is measured once. Accordingly, the blood analysis device D may decrease in volume and be simply stored and used. The blood analysis device D may be free from pollution. The blood analysis device D may exactly measure various bio-markers even by using a small amount of blood. The blood analysis device D may be provided at an inexpensive price.

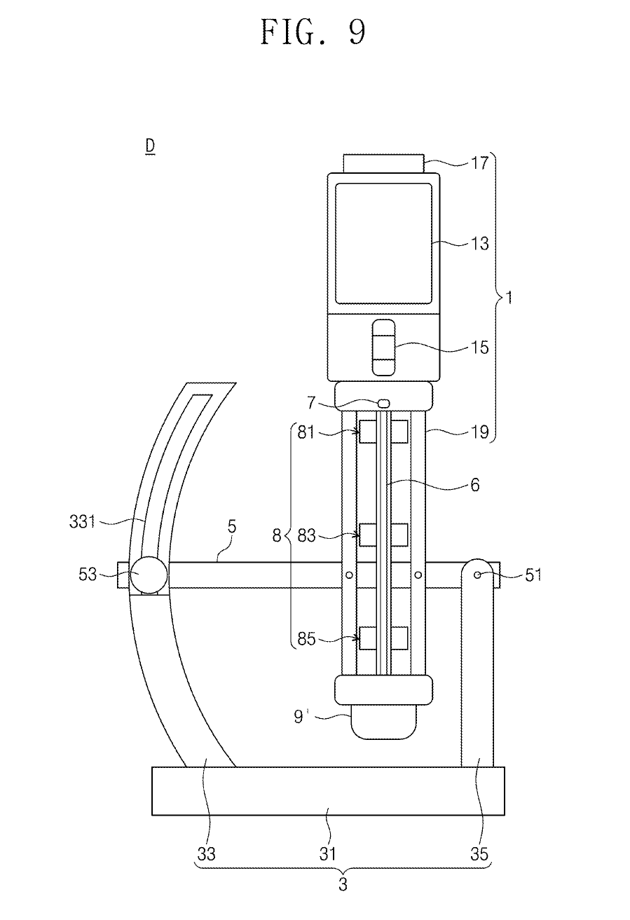

FIG. 9 is a front view illustrating a blood analysis device according to another embodiment of the inventive concept. For convenience of description, the substantially same or similar components as those described with reference to FIGS. 1 to 8 will be omitted.

Referring to FIG. 9, a blood analysis device D may further include an absorbent agent 9′. The absorbent agent 9′ may be coupled to one end of the capillary tube 6. The absorbent agent 9′ may be coupled at the position of the absorption sensor 9 described with reference to FIGS. 1 to 8. The absorbent agent 9′ may be a porous medium. The absorbent agent 9′ may absorb the blood B. The moving speed of the blood B flowing in the capillary tube 6 may be adjusted by the absorbent agent 9′. The blood B in the capillary tube 6 may move by an absorption force of the absorbent agent 9′, the gravity applied to the blood B, and the capillary phenomenon of the capillary tube 6.

When the absorbent agent 9′ is used, an additional pump may be unnecessary. The absorbent agent 9′ may be inexpensive. Accordingly, when the absorbent agent 9′ is used, the blood analysis device D may become inexpensive. The absorbent agent 9′ may have a small volume. Accordingly, when the absorbent agent 9′ is used, the blood analysis device D may become smaller in volume.

According to the blood analysis device of the embodiment of the inventive concept, the physical/chemical/biological characteristics of the blood may be measured at once.

According to the blood analysis device of the embodiment of the inventive concept, various bio-markers of the blood may be exactly and rapidly measured while the volume of the blood analysis device decreases.

The objects of the present disclosure are not limited to the aforementioned object, but other objects not described herein will be clearly understood by those skilled in the art from descriptions below.

Although the exemplary embodiments of the present invention have been described, it is understood that the present invention should not be limited to these exemplary embodiments but various changes and modifications can be made by one ordinary skilled in the art within the spirit and scope of the present invention as hereinafter claimed.

What is claimed is:

1. A blood analysis device comprising:
a capillary tube;
photo sensors disposed on a sidewall of the capillary tube to detect blood flowing in the capillary tube; and
an absorption sensor coupled to one end of the capillary tube to absorb the blood in the capillary tube,
wherein the absorption sensor contains a sensing material,
the photo sensors include a first photo sensor and a second photo sensor, and
each of the first photo sensor and the second photo sensor comprises a light emitting part emitting light having wavelengths different from each other.

2. The blood analysis device of claim 1, wherein the sensing material configured to measure chemical or biological characteristics.

3. The blood analysis device of claim 2, further comprising:
a control unit; and
a reader configured to detect variation of the absorption sensor,
wherein the reader transmits information on the variation of the absorption sensor to the control unit.

4. The blood analysis device of claim 1, wherein the photo sensors measure a transmittance of the blood flowing in the capillary tube.

5. The blood analysis device of claim 1, further comprising a control unit,
wherein the control unit receives information on a transmittance of the blood from the photo sensors to calculate a viscosity of the blood flowing in the capillary tube.

6. The blood analysis device of claim 5, further comprising a temperature sensor,
wherein the control unit receives information on a temperature of the blood from the temperature sensor to correct the viscosity of the blood.

7. The blood analysis device of claim 1, further comprising:
a rotation guide; and
a rotation support,
wherein the capillary tube is coupled to the rotation support so as to rotate along the rotation guide.

8. The blood analysis device of claim 5, further comprising a display unit,
wherein the control unit displays the viscosity of the blood on the display unit.

9. The blood analysis device of claim 1, wherein the capillary tube contains a transparent material.

10. The blood analysis device of claim 1, wherein the capillary tube has an inner surface that is surface-treated by EDTA or heparin.

11. The blood analysis device of claim 1, wherein the capillary tube has an inside diameter of about 1 mm or less.

12. A blood analysis device comprising:
a capillary tube;
a first photo sensor;
a second photo sensor;
an absorption sensor coupled to one end of the capillary tube to absorb the blood in the capillary tube; and
a control unit,
wherein each of the first photo sensor and the second photo sensor is disposed on a sidewall of the capillary tube to measure a transmittance of blood flowing in the capillary tube,
the control unit receives information on the transmittance of the blood from the first and second photo sensors to calculate hematocrit (HCT) of the blood flowing in the capillary tube, and
the absorption sensor contains a sensing material configured to measure chemical or biological characteristics.

13. The blood analysis device of claim 12, wherein the control unit receives the information on the transmittance of the blood from the first and second photo sensors to calculate a viscosity of the blood flowing in the capillary tube.

14. The blood analysis device of claim 12, wherein the first photo sensor comprises a first light emitting part and a first light receiving part,
the second photo sensor comprises a second light emitting part and a second light receiving part, and
the first light emitting part and the second light emitting part emit light having wavelengths different from each other.

15. The blood analysis device of claim 14, wherein the first light emitting part emits first light,
the second light emitting part emits second light,
the first light has a wavelength of about 800 to about 1000 nm, and
the second light has a wavelength of about 500 to about 600 nm.
